(19) United States
(12) Patent Application Publication
(10) Pub. No.: US 2026/0022341 A1
(43) Pub. Date: Jan. 22, 2026

(54) METHOD OF GENERATING A HOMOCELLULAR PROGENITOR CELL CULTURE FROM A HETEROCELLULAR TISSUE SAMPLE

(71) Applicant: MOSA MEAT B.V., Maastricht (NL)

(72) Inventors: Joshua Edwin FLACK, Maastricht (NL); Tobias MESSMER, Maastricht (NL); Rui Horst HÜBER, Maastricht (NL); Lieke Maria Louisa SCHAEKEN, Maastricht (NL); Beatriz DA SILVA LOPES MARTINS, Maastricht (NL); Richard Gerardus Johannes DOHMEN, Maastricht (NL)

(73) Assignee: MOSA MEAT B.V., Maastricht (NL)

(21) Appl. No.: 18/993,733

(22) PCT Filed: Jul. 14, 2023
(86) PCT No.: PCT/NL2023/050380

§ 371 Date: Jan. 13, 2025

(30) Foreign Application Priority Data
Jul. 15, 2022 (NL) 2032514

(51) Int. Cl. C12N 5/077

(52) U.S. Cl. CPC C12N 5/0658; C12N 2500/25; C12N 2500/32; C12N 2501/105; C12N 2501/115; C12N 2501/12; C12N 2501/135; C12N 2501/165; C12N 2501/2306; C12N 2509/00; C12N 2533/52

(57) ABSTRACT

The present disclosure is in the field of culturing a homocellular cell culture of progenitor cells form a heterocellular tissue sample. The present disclosure relates to a method for specifically generating muscle progenitor cells or adipogenic progenitor cells from an isolated tissue sample, preferably a muscle tissue sample, e.g., from mammalian origin, preferably from bovine, ovine, murine, or porcine origin. The method includes culturing a heterocellular tissue sample comprising a muscle progenitor cell and an adipogenic progenitor cell in a selection medium including comprsing at least one compound which specifically promotes the proliferation of muscle progenitor cells or of adipogenic progenitor cells leading to a homocellular cell culture. Further, the present disclosure relates to the selection medium which facilitates selective generation of muscle progenitor cells or of adipogenic progenitor cells. The method and selection medium of the invention can be used for producing homocellular cell cultures of muscle progenitor cells or of adipogenic progenitor cells. The homocellular cell cultures produced according to the present disclosure can be used to produce a cell culture-based meat product, preferably for animal or human consumption.

### FIELD

The aspects of the disclosed embodiments is in the field of culturing a homocellular cell culture of progenitor cells form a heterocellular tissue sample. The disclosed embodiments relate to a method for specifically generating muscle progenitor cells or adipogenic progenitor cells from an isolated tissue sample, preferably a muscle tissue sample, e.g., from mammalian origin, preferably from bovine, ovine, murine, or porcine origin. The method comprises culturing a heterocellular tissue sample comprising a muscle progenitor cell and an adipogenic progenitor cell in a selection medium comprising at least one compound which specifically promotes the proliferation of muscle progenitor cells or of adipogenic progenitor cells leading to a homocellular cell culture. Further, the disclosed embodiments relate to the selection medium which facilitates selective generation of muscle progenitor cells or of adipogenic progenitor cells. The method and selection medium of the disclosed embodiments can be used for producing homocellular cell cultures of muscle progenitor cells or of adipogenic progenitor cells. The homocellular cell cultures produced according to the present disclosure can be used to produce a cell culture-based meat product, preferably for animal or human consumption.

### BACKGROUND

In vitro culturing of animal cells requires the use of complex cultivation media providing essential nutrients, vitamins, growth factors, proteins, electrolytes, etc. which ensure the survival and proliferation of the cells in culture. A wide variety of cultivation media are known from the art which promote the proliferation of animal cells in culture. Cultivation media of the state of the art have been shown to facilitate cultivation and proliferation of diverse cell types at the same time. This means that most cultivation media are suitable for culturing diverse cell types. For example, WO 2021/158103 provides a serum-free cultivation medium which facilitates cultivation of both muscle progenitor cells and adipogenic progenitor cells.

The fact that such cultivation media support the growth of a number of different cell types in parallel does not represent a problem when cultivation is initiated either from a single cell or from a homocellular primary culture. In these cases a single cell type will be grown and the same cultivation media can be used to grow different cell types separately.

This is different in the present disclosure. The starting material is a mixture of different cell types and of cells which are able to differentiate into different cell types depending on the growth conditions. Such mixture of cells can for example be obtained in a biopsy. If this mixture of different cell types is grown in a “standard” cultivation medium all cell types of the mixture will grow in parallel. The specific growth of one selected cell type is only possible, if conditions are selected allowing the growth of a specific cell type.

Culturing and generating homocellular cell cultures from heterocellular tissue samples is for example desired in industrial bioprocesses, e.g. for cell culture-based meat production, in order to guarantee quality standards and to achieve required characteristics of the product to be produced. Here the ability of known cultivation media to support the survival and proliferation of different cell types represents a particular problem. As a result, in such processes the different cell types first need to be carefully separated from each other and cultured independently in order to obtain homocellular cell cultures. However, the separation of cell types represents an additional time-and cost consuming process step. Moreover, the applied separation methods, such as antigen-based cell sorting (e.g., FACS) may not result in pure homocellular cultures. After sorting, the cell cultures may still be heterogeneous and comprise contaminating cell types. Culturing those contaminated and thus, still heterocellular cell cultures in a cultivation medium of the state of the art results in the proliferation not only of the desired cell type, but also of the contaminating cells. In the case of those contaminating cell type(s) exhibiting a higher proliferation rate than the desired cell type, they may even overgrow the desired cell type. As a consequence, homocellular cell cultures are difficult to achieve. Due to the strict quality and safety requirements in industrial controlled bioprocesses heterocellular cell cultures are preferably avoided. This brings about the drawbacks of excessive production times and undue costs in controlled bioprocesses, e.g., in the production of cell culture-based food.

The current state of the art lacks cultivation methods which facilitate the generation of a homocellular cell culture when culturing and proliferation is initiated from a heterocellular cell culture, e.g., a tissue sample. Hence, there is a need in the art for reliable cultivation methods and means to obtain a homogeneous cell culture from culturing heterocellular tissue samples. Especially, there is a need in the art to establish a cost and time efficient method for generating homocellular progenitor cell cultures such as homocellular cell cultures of muscle progenitor cells or of adipogenic progenitor cells from heterocellular tissue samples. Such a method is especially desirable since a homocellular progenitor cell culture is a prerequisite for a controlled cell differentiation which is for example essential in the production of a cell culture-based meat product.

The aspects of the disclosed embodiments aim to solve the problem of contaminated cell cultures derived from heterocellular tissue samples and provides a method and medium for generating homocellular cell cultures of muscle progenitor cells or of adipogenic progenitor cells from heterocellular tissue samples.

### SUMMARY OF THE DISCLOSED EMBODIMENTS

The present disclosure refers to a method for specifically generating a homocellular cell culture of muscle progenitor cells or of adipogenic progenitor cells from a heterocellular muscle tissue sample comprising the steps of:
a) Providing a muscle tissue sample comprising at least one muscle progenitor cell and at least one adipogenic progenitor cell;
b) Culturing the muscle tissue sample in a selection medium to specifically proliferate the at least one muscle progenitor cell or the at least one adipogenic progenitor cell, wherein the selection medium comprises at least one compound which specifically promotes the proliferation of the at least one muscle progenitor cell or of the at least one adipogenic progenitor cell, wherein the at least one compound which specifically promotes the proliferation of the at least one muscle progenitor cell is selected from the group consisting of fibroblast growth factor 2(FGF-2), hepatocyte growth factor (HGF), Triiodothyronine (T3), Dexamethasone, Hydrocortisone, Indomethacin, SB203580 (4-{4-(4-Fluorophenyl)-2-[4-(methanesulfinyl) phenyl]-1H-imidazol-5-yl} pyridine), Vitronectin, Laminin 521, and a combination thereof, or wherein the selection medium comprises at least one compound which specifically promotes the proliferation of the at least one adipogenic progenitor cell wherein the at least one compound is selected from the group consisting of PDGF-bb, Interleukin 6 (IL-6), Fibronectin, Collagen, and a combination thereof; and
c) Optionally, harvesting the generated homocellular cell culture.

The method of the present disclosure optionally further comprises the steps of:
a-1) enzymatically pre-processing the heterocellular muscle tissue sample for obtaining a heterocellular cell mixture, and/or
a-2) pre-sorting the muscle progenitor cell and/or the adipogenic progenitor cell of the heterocellular muscle tissue sample, wherein both steps are performed prior the culturing of step b). The pre-sorting of step a-2) is for example performed by antigen-based cell sorting, preferably by fluorescence activated cell sorting (FACS).

The homocellular cell culture of muscle progenitor cells or of adipogenic progenitor cells prepared by a method of the present disclosure for example have a percentage of muscle progenitor cells or of adipogenic progenitor cells from 90% to 100%, preferably from 95% to 100%, more preferably above 97% referring to the total number of cells. The percentage of muscle progenitor cells or of adipogenic progenitor cells obtained by the method of the disclosed embodiments is for example determined in the proliferation phase.

At least one compound which specifically promotes the proliferation of the muscle progenitor cell is for example selected from the group consisting of fibroblast growth factor 2 (FGF-2), hepatocyte growth factor (HGF), Triiodothyronine (T3), Dexamethasone, Hydrocortisone, Indomethacin, SB203580 (4-{4-(4-Fluorophenyl)-2-[4-(methanesulfinyl) phenyl]-1 H-imidazol-5-yl} pyridine), Vitronectin, a laminin such as Laminin 521, and a combination thereof. For example, the compounds for specifically promoting the proliferation of the muscle progenitor cell comprise HGF, for example 10xHGF (e.g. about 50 ng/ml), and Triiodothyronine (T3) (e.g. about 30 nM).

At least one compound which specifically promotes the proliferation of the adipogenic progenitor cell is for example selected from the group consisting of PDGF-bb, Interleukin 6 (IL-6), Fibronectin, Collagen, and a combination thereof.

Further, the at least one compound of the present disclosure is for example comprised in the selection medium in solution or attached to a support structure. For example, the at least one compound is coated on a culture vessel.

The culturing of step b) according to the method of the present disclosure optionally comprises culturing the muscle tissue sample in a culture vessel which is coated with at least one of the compounds specifically promoting the proliferation of the muscle progenitor cell or of the adipogenic progenitor cell. The culturing of step b) is for example performed using a selection medium comprising HGF, T3 and a laminin such as Laminin 521, preferably about 50 ng/ml HGF, about 30 nM T3 and Laminin 521, for specifically proliferating muscle progenitor cells, wherein the laminin such as Laminin 521 is coated on a culture vessel.

The muscle progenitor cell is for example a myosatellite cell (SC) and/or the adipogenic progenitor cell is for example a fibro-adipogenic progenitor cell (FAP).

Further, the heterocellular muscle tissue sample is for example from mammalian, preferably from bovine, ovine, murine, or porcine origin, more preferably from bovine origin.

The method of the present disclosure is for example for producing a cell culture-based meat product, preferably for animal or human consumption.

Further, the aspects of the disclosed embodiments are is directed to a selection medium for culturing a muscle tissue sample comprising a muscle progenitor cell and an adipogenic progenitor cell, wherein the selection medium comprises at least one compound which specifically promotes the proliferation of the muscle progenitor cell or of the adipogenic progenitor cell.

In the selection medium of the aspects of the disclosed embodiments the at least one compound specifically promoting the proliferation of the muscle progenitor cell is for example selected from the group consisting of fibroblast growth factor 2 (FGF-2), hepatocyte growth factor (HGF), Triiodothyronine (T3), Dexamethasone, Hydrocortisone, Indomethacin, SB203580, Vitronectin, Laminin 521, and a combination thereof. Optionally, the selection medium comprises HGF and T3, preferably about 50 ng/ml HGF and about 30nM T3, for specifically promoting the proliferation of the muscle progenitor cell.

In the selection medium of the aspects of the disclosed embodiments, the at least one compound specifically promoting the proliferation of the adipogenic progenitor cells is for example selected from the group consisting of PDGF-bb, Interleukin 6 (IL-6), Fibronectin, Collagen, and a combination thereof.

The at least one compound comprised in the selection medium is for example in solution or attached to a support structure, e.g., coated on a culture vessel.

Optionally, the selection medium of the aspects of the disclosed embodiments is serum-free.

The selection medium of the aspects of the disclosed embodiments is for example used for specifically generating a homocellular cell culture of muscle progenitor cells or of adipogenic progenitor cells from a heterocellular muscle tissue sample, preferably from a heterocellular bovine muscle tissue sample.

### DESCRIPTION OF THE DRAWINGS

FIG. 1: Expression profiling of SCs.

Volcano plot highlighting differentially expressed (DE) genes significantly upregulated in SCs (right-hand side, light grey) and FAPs (left-hand side, dark grey).

FIG. 2: Identification of compounds to favor SC or FAP growth.

(A) Bar graphs showing the increase (bars filled with points) or decrease (bars filled with lines) of SCs in a cell culture consisting of SCs and FAPs after one passage compared to the control for Hepatocyte Growth factor; HGF (0×=0 ng/ml; 10×=50 ng/ml instead of 5 ng/ml); PDGF-bb (0×=0 ng/ml; 10×=50 ng/ml); Indomethacin, IC (50 μM); Hydrocortisone, HC (138 nM); Triiodothyronine, T3 (30 nM); DAPT (5 μM); Fibroblast growth factor 2, FGF2 (1×=10 ng/ml, 10×=100 ng/ml); IL6 (20 ng/ml). (B) Bar graphs showing the increase of SCs in a cell culture consisting of SCs and FAPs after one passage compared to control and DMSO control for DAPT (10 μM) and SB203580 (10 μM). The ratio of SCs in the cell culture consisting of SCs and FAPs was normalized against a control ratio (Ctrl; set at 1) which means that a promotion of SCs results in an increase of SCs compared to the control cell culture (>1), whereas a relative decrease of SCs compared to the control cell culture (<1) denotes at the same time for a promotion of FAP proliferation in the cell culture.

FIG. 3: Coating conditions favor specific growth of SC or FAP in long-term proliferation. Percentage of SCs in total cells (consisting of SCs and FAPs) when subjected to culturing with different compounds applied as coatings over the course of long-term proliferation. Vitronectin and laminin 521 beneficially allow for maintaining high percentages of SCs during proliferation. Collagen and fibronectin favor proliferation of FAPs.

FIG. 4: Culture conditions favor specific growth of SC or FAP in long-term proliferation. (A) Percentage of SCs in control media culture, consisting of SCs and FAPs, and with single and combined media improvements over the course of long-term proliferation. (B) Growth curve of bovine SCs in control media culture, consisting of SCs and FAPs, with single and combined media improvements, presented as population doublings (PDs) against passage. (C) Growth curve of bovine FAPs in control media culture, consisting of SCs and FAPs, with single and combined media improvements presented as PDs against passage. The passages in FIGS. 4A to 4C correspond to the same experiment. Thus, the growth of SCs represented in FIG. 4B together with the growth of FAPs represented in FIG. 4C results in the ratio/percentage of SCs represented in FIG. 4A.

### DETAILED DESCRIPTION OF THE DISCLOSED EMBODIMENTS

The present disclosure provides a method for selectively generating a homocellular cell culture of muscle progenitor cells or of adipogenic progenitor cells from a heterocellular tissue sample, preferably a muscle tissue sample, comprising the steps of providing a tissue sample comprising a muscle progenitor cell and an adipogenic progenitor cell and culturing the tissue sample in a selection medium to specifically proliferate the muscle progenitor cell or the adipogenic progenitor cell. The selection medium used for culturing comprises at least one compound which specifically promotes the proliferation of the muscle progenitor cell or of the adipogenic progenitor cell. Further, the aspects of the disclosed embodiments relates to a selection medium for culturing a tissue sample, preferably a muscle tissue sample, comprising a muscle progenitor cell and an adipogenic progenitor cell, wherein the selection medium comprises at least one compound which specifically promotes the proliferation of the muscle progenitor cell or of the adipogenic progenitor cell. The present disclosure refers also to the use of the selection medium for specifically generating homocellular cell cultures of muscle progenitor cells or of adipogenic progenitor cells from a tissue sample, preferably a muscle tissue sample as well as to the use of homocellular cell cultures for producing a cell culture-based meat product.

Surprisingly, the aspects of the disclosed embodiments facilitates the selective generation of a homocellular cell culture of muscle progenitor cells or of adipogenic progenitor cells from a heterocellular tissue sample, preferably a muscle tissue sample. Thus, the aspects of the disclosed embodiments overcome the disadvantage of laborious separation processes and/or of wasting production resources due to cell contamination.

In the following, the features of the aspects of the disclosed embodiments will be described in more detail. It should be understood that embodiments may be combined in any manner and in any number to create additional embodiments. The variously described examples and embodiments should not be construed to limit the present disclosure to only the explicitly described embodiments. This description should be understood to support and encompass embodiments which combine the explicitly described embodiments with any number of the disclosed features. Furthermore, any permutations and combinations of all described features in this application should be considered disclosed by the description of the present application unless the context indicates otherwise.

Throughout this specification and the claims, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the disclosed embodiments (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the present disclosure and does not pose a limitation on the scope of the disclosed embodiments otherwise claimed. No language in the specification should be construed as indicating any non-claimed element essential to the practice of the disclosed embodiments.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the disclosed embodiments. More specifically, all referenced documents are incorporated by reference to the same extent as if each individual document was specifically and individually indicated to be incorporated by reference.

### Definitions

The term “culturing”, as used herein, includes reference to the propagation and/or proliferation (expansion) of cells such as progenitor cells. In the context of the disclosed embodiments, this term preferably includes reference to the proliferation and therefore expansion of muscle progenitor cells or adipogenic progenitor cells from a heterocellular tissue sample, such as a muscle tissue sample. The selection medium of the aspects of the disclosed embodiments can be employed to proliferate (expand) muscle progenitor cells, such as myosatellite cells (SCs), or adipogenic progenitor cells, such as fibro-adipogenic progenitor cells (FAPs), from any mammalian origin, such as bovine, ovine, murine, porcine, or human origin.

The terms “proliferation” and “expansion”, as used herein, can be used interchangeably. These terms include reference to increasing the population size of muscle progenitor cells, such as SCs, or adipogenic progenitor cells, such as FAPs, in cell culture, i.e. progenitor cells are generating other progenitor cells by cell proliferation. Such an expanded progenitor cell population can subsequently be cultured in a differentiation medium for differentiating said progenitor cells into differentiated or partially differentiated muscle cells or fat adipocytes/cells that can be incorporated into a cell-culture based meat product for animal or human consumption. However, first, before differentiation, sufficient amounts of muscle and/or adipocyte progenitor cells, e.g. from bovine origin, need to be produced by proliferation/expansion. Preferably, the muscle and/or adipocyte progenitor cells are cultured and proliferated according to the present disclosure in order to obtain homocellular cell cultures of both cell types. Preferably, the muscle and/or adipocyte progenitor cells are cultured and proliferated according to the present disclosure in order to specifically promote the proliferation of muscle or adipocyte progenitor cells. Specific proliferation is for example obtained in a culture medium that contains one or more compounds which promote the proliferation of either muscle or adipocyte progenitor cells. The proliferation is for example promoted by the addition of one or more compounds which accelerate the proliferation of the favored cell type and/or by the addition of one or more compound which delay the proliferation of the one or more non-favored cell types. For example, the specific promotion of either muscle or adipocyte progenitor cells can be obtained by one or more compounds which accelerate the proliferation of the favored cell type (muscle or adipocyte progenitor cells). For example, the specific promotion of either muscle or adipocyte progenitor cells can be obtained by one or more compound which delays the proliferation of the non-favored cell types (muscle or adipocyte progenitor cells). For example, the specific promotion of muscle progenitor cells can be obtained by one or more compounds which accelerate the proliferation of muscle progenitor cells. For example, the specific promotion of muscle progenitor cells can be obtained by one or more compounds which delay the proliferation of adipocyte progenitor cells. For example, the specific promotion of adipocyte progenitor cells can be obtained by one or more compounds which accelerate the proliferation of adipocyte progenitor cells. For example, the specific promotion of adipocyte progenitor cells can be obtained by one or more compounds which delay the proliferation of muscle progenitor cells.

The term “mammalian”, as used herein, includes reference to any animal of the class Mammalia. Non-limiting examples of animals belonging to the class Mammalia are cattle, pigs, sheep, deer, and mice. A mammalian cell includes cells isolated from, derived from, differentiated from, expanded or originally found in an animal of the class Mammalia, and also includes cultured cells. A preferred mammal is a Bos taurus.

The term “adipocyte”, as used herein, can be used interchangeably with the term “fat cell” and includes reference to a cultured fat cell or adipocyte or cultured fat tissue. Adipocytes may be categorized as forming white adipose tissue or brown adipose tissue. Adipocytes are found throughout the body. Adipocytes synthesize and store fat, including but not limited to lipids and triglycerides.

The term “progenitor cell” as used herein, includes reference to a cell that is able to differentiate into a more specialized cell. The term “progenitor cell” can be used interchangeably with the term “precursor cell”. Progenitor cells are for example stem cells, myosatellite cells (SCs), fibro-adipogenic progenitor cells (FAPs), intermediate progenitor cells, radial glial cells, bone marrow stromal cells, periosteum, pancreatic progenitor cells, angioblasts, blast cells, adipogenic progenitor cells or fibro-adipogenic progenitor cells, preferably myosatellite cells (SCs) or fibro-adipogenic progenitor cells (FAPs). No limitation to the stage of development is intended.

The term “muscle progenitor cell”, as used herein, can be used interchangeably with the term “muscle stem cell” or “myogenic progenitor (cell)”. These terms include reference to adult stem cells, present in tissue such as skeletal muscle tissue, which are usually multipotent and which can self-renew and are capable of giving rise to muscle cells such as skeletal muscle cells. The term “muscle progenitor cell” can also be referred to as “muscle cell progenitor”. A preferred muscle progenitor cell is a bovine muscle progenitor cell such as a bovine myosatellite cell. For example, the muscle progenitor cell, or the myosatellite cell, is a (skeletal) muscle tissue-derived progenitor cell. Such a cell can be obtained by direct isolation from an animal or can be obtained after proliferation (expansion) in a proliferation medium. Such a cell can be genetically modified or not genetically modified, preferably not genetically modified. Progenitor cells from the muscle can be isolated based on their positive expression of CD29 as previously described (Ding et al., Sci. Rep. 17(8): 10808 (2018)). Such progenitor cells can be expanded in population size by using a cultivation medium, preferably the selection medium according to the present disclosure.

The terms “myosatellite cell” or “satellite cell”, as used herein interchangeably and abbreviated as “SC”, include reference to a small multipotent cell and can be found in mature muscle tissue. Myosatellite cells are precursors to skeletal muscle cells, able to differentiate into skeletal muscle cells. They are precursor cells that can be obtained from muscle tissue. They have the potential to provide additional myonuclei to their parent muscle fiber, or return to a quiescent state. More specifically, upon activation, satellite cells can re-enter the cell cycle to proliferate or differentiate into myoblasts and ultimately into myocytes which will fuse forming myotubes. Myosatellite cells are generally located between the basement membrane and the sarcolemma of muscle fiber. Myosatellite cells generally express a number of distinctive genetic markers. Most satellite cells express PAX7 and PAX3. The myosatellite cell is for example a bovine myosatellite cell.

The term “adipogenic progenitor cell”, as used herein, includes reference to a cell that is able to differentiate into an adipocyte. Adipogenic progenitor cells are for example pluripotent stem cells, induced pluripotent stem cells (iPSCs), mesenchymal stem cells, fibro-adipogenic progenitor (FAP) cells, lipoblasts, adipoblasts and preadipocytes. The adipogenic progenitor cell can for instance be derived from muscle or fat, and is in embodiments a muscle-derived adipogenic progenitor cell such as a (skeletal) muscle-derived fibro-adipogenic progenitor (FAP) cell, which is a type of adult stem cell. Preferably, the adipogenic progenitor cell is a mammalian adipogenic progenitor cell, for instance a bovine, ovine, porcine or murine, preferably bovine, adipogenic progenitor cell. FAP cells can be purified from muscle tissue samples by methods involving antigen-based cell sorting such as FACS. Cell surface markers expressed by bovine FAPs are CD9, CD14, CD49e (ITGA5), CD61 (ITGB3), CD140a (PDGFRA), and ITGA9. Bovine FAPs lack expression of hematopoietic marker CD45, endothelial marker CD321 (F11R), and the myogenic progenitor markers CD56 (NCAM1) and ITGA7. Preferably, the FAP cell is a bovine FAP cell.

The term “serum-free”, as used herein, includes reference to a culture medium, preferably to the selection medium of the present disclosure that is formulated in the absence of serum such as human serum, bovine serum (also known as FBS, or fetal bovine serum; or fetal calf serum), horse serum or cell lysates of cells in said serum. A serum-free medium may contain serum proteins by way of supplementation of said serum protein such as serum albumin to said medium. However, preferably, all components of said medium are animal-free, i.e., that the components are not obtained from an animal, but are for instance recombinantly produced. Preferably, a serum-free medium as disclosed herein is a chemically defined medium, i.e., a medium that is defined by the presence and/or absence of specific components. The concentrations of the components of a serum-free medium as disclosed herein can routinely be adjusted and optimized for the culturing and differentiation of muscle progenitor cells such as mammalian muscle progenitor cells, e.g., bovine, ovine or porcine muscle progenitor cells or for the culturing and differentiation of adipogenic progenitor cells such as mammalian adipogenic progenitor cells, e.g., bovine, ovine or porcine adipogenic progenitor cells. A non-limiting example of a serum-free medium for proliferation or expansion of muscle progenitor cells and/or adipogenic progenitor cells is the medium disclosed in WO 2021/158103. For instance, the at least one compound which specifically promotes the proliferation of the at least one muscle progenitor cell or the at least one compound which specifically promotes the proliferation of the at least one adipogenic progenitor cell is comprised in (or supplemented to) a serum-free medium that comprises an albumin (e.g. a human serum albumin), L-Ascorbic acid 2-phosphate, a-linolenic acid, insulin, transferrin, sodium selenite, ethanolamine, L-alanyl-L-glutamine or glutamine, IGF1, VEGF, and a (e.g. DMEM/F12) basal medium. For instance, such a serum free medium comprises albumin (5mg/ml); L-Ascorbic acid 2-phosphate (50 or 155 μg/ml); α-linolenic acid (1 μg/ml); insulin (10 μg/ml), transferrin (5.5 μg/ml), sodium selenite (0.0067 μg/ml), ethanolamine (2 μg/ml) (or ITSE, 1%); L-alanyl-L-glutamine or glutamine (2 mM); IGF1 (100 ng/ml); VEGF (10 ng/ml); and DMEM/F12 basal medium. Somatotropin may also be included in said selection medium. Glucose and/or Penicillin/Streptomycin/Amphotericin (PSA) may also be included in said selection medium.

The term “bovine”, as used herein, includes reference to any member of the subfamily Bovinae. The subfamily Bovinae includes the tribes Boselaphini, Bovini and Tragelaphini. Preferably, bovine as disclosed herein refers to the members of the subfamily Bovinae that are used for animal, preferably human, consumption. Non-limiting examples of such members include domestic cattle (Bos taurus and subspecies Bos taurus taurus; Bos taurus indicus), banteng (Bos javanicus), gayal or mithun (Bos frontalis), gaur (Bos gaurus), yak (Bos grunniens; Bos mutus), water buffalo (Bubalus arnee; Bubalus bubalis), American bison (Bison bison), kudu (Tragelaphus strepsiceros; Tragelaphus imberbis), common eland (Taurotragus oryx), giant eland (Taurotragus derbianus), and nilgai (Boselaphus tragocamelus). Especially preferred bovine species as disclosed herein are Bos taurus and its subspecies.

The term “ovine”, as used herein, includes reference to any member of the genus Ovis. Preferably, ovine as disclosed herein refers to the members of the genus Ovis that are used for animal, preferably human, consumption. A non-limiting example of such members includes domestic sheep (Ovis aries).

The term “porcine”, as used herein, includes reference to any member of the genus Sus. Preferably, porcine as disclosed herein refers to the members of the genus Sus that are used for animal, preferably human, consumption. Non-limiting examples of such members include domestic pig (Sus scrofa domesticus) and wild boar (several Sus scrofa subspecies).

The term “murine”, as used herein, includes reference to any member of the family Muridae. Preferably, murine as disclosed herein refers to the members of the family Muridae that are used for animal consumption. Non-limiting examples of such members include mice and rats.

The term “meat product”, as used herein, includes reference to cell culture based meat that is suitable for animal, preferably human, consumption. A meat product generally comprises muscle tissue and preferably also fat tissue. A cell culture based meat product may for example, amongst others, be distinguished from a natural animal-derived meat product by the absence of for instance immune cells such as antigen-presenting cells (APCs) such as monocytes or macrophages, T cells and B cells. Other distinguishing characteristics are for example the absence of immune cell effector molecules such as antibodies, or red blood cells. Other distinguishing characteristics may for example be the absence of cartilage tissue, lower levels of fibrous tissue and/or absence of antibiotics and/or antibiotic residues. In some cases, the cell culture-based meat product may be indistinguishable from natural animal-derived meat. The terms “cell culture-based meat product”, “cultured meat (product)”, and “cellular agriculture” are used interchangeably.

The term “heterocellular”, as used herein, refers to mixtures, cultures, populations, or samples comprising cells of different cell types, i.e., where the cells are heterogeneous. Such cell cultures may also be called “heterocultures”.

The term, “homocellular”, as used herein, refers to mixtures, cultures, populations, or samples comprising cells of mainly one cell type only, i.e., where the cells are homogeneous. Such cell cultures may also be called “monocultures”, i.e., a cell culture predominantly consisting of one specific cell type. The percentage of the determining cell type in a homocellular cell cultures/monoculture is for example from 90-100%, 92-99.9%, 93-99.7%, 94-99.5%, 95-99%, above 90%, above 93%, above 95%, above 97%, above 98%, above 99%, or above 99.5% referring to the total number of cells. The percentage is for example determined in the proliferation phase of the cell culture.

The present disclosure provides a method for specifically generating a homocellular cell culture of muscle progenitor cells, such as SCs, or of adipogenic progenitor cells, such as FAPs, from a heterocellular tissue sample. The tissue sample may be derived from any kind of animal tissue comprising a muscle progenitor cell and an adipogenic progenitor cell. For example, the sample is derived from muscle tissue. The tissue sample may be derived from any animal species. The tissue sample is for example from mammalian origin, e.g., from bovine, ovine, murine, or porcine origin.

The tissue sample, preferably muscle tissue sample, is for example derived from a cadaver, or from a living animal. For example the tissue sample is derived form a wildtype animal. Optionally, the cells comprised in the tissue sample are not genetically modified.

The tissue sample, preferably muscle tissue sample, can be provided by taking a biopsy on an animal. Any other method for obtaining a tissue sample known in the art can be used for the method of the present disclosure.

The muscle progenitor cell comprised in the tissue sample may be a myosatellite cell (SC). The adipogenic progenitor cell may be a mesenchymal stem cell, fibro-adipogenic progenitor (FAP) cell, lipoblast, adipoblast and preadipocyte. For example, the adipogenic cell comprised in the tissue sample may be a fibro-adipogenic progenitor cell (FAP).

### Pre-Processing of the Tissue Sample

The method of the present disclosure may comprise pre-processing of a tissue sample, preferably muscle tissue sample. Pre-processing may be applied in order to obtain a heterocellular cell mixture, preferably a heterocellular mononucleated cell mixture. The pre-processing for example comprises the removal of excess fat and fibrous tissue if present. For example, removal of excess fat and fibrous tissue is performed chopping the tissue sample with scissors.

For example, the tissue sample, preferably the muscle tissue sample, is enzymatically pre-processed, e.g., by subjecting the sample to enzymatic digestion, for instance by using a matrix metalloproteinase, such as a collagenase. This allows for dissociation of muscle fibers. For example, collagenase AFC A (Worthington, CLS-1, 2000 U/ml) is used. Exemplary conditions for dissociation using collagenase are an incubation time of 45 minutes and an incubation temperature of 37° C. The muscle tissue sample may be incubated with erythrocyte lysis buffer. For example, 1× ACK erythrocyte lysis buffer is used. Exemplary incubation conditions for erythrocyte lysis buffer are an incubation time of 1 minute and an incubation temperature of 37° C.

Cells obtained from the tissue sample, preferably muscle tissue sample, after said enzymatic digestion and/or said incubation with erythrocyte lysis buffer are for example pre-cultured by resuspending and incubating said cells in serum-free proliferation medium as disclosed herein (and in WO 2021/158103), and e.g. seeded into bovine collagen type I coated tissue plates. The collagen type I used for coating said tissue plates is for example C2124 from Sigma, and can be applied at 2.5 g/cm². Preferably, incubation in the serum-free proliferation medium is done at 37° C.

### Pre-Sorting

The method of the disclosed embodiments may also comprise a step of pre-sorting of cell types comprised in the tissue sample, preferably muscle tissue sample. For example, muscle progenitor cells, such as SCs, and/or adipogenic progenitor cells, such as FAPs, of the heterocellular (muscle) tissue sample are pre-sorted. The pre-sorting is for example performed after the pre-processing of the (muscle) tissue sample.

Pre-sorting may be performed by antigen-based cell sorting such as fluorescence activated cell sorting (FACS). Pre-sorting is for example done using a MACSQuant Tyto sorter from Miltenyi.

For example, FAPs are characterized by the presence of cell surface markers CD9, CD14, CD49e, CD61, CD140a, ITGA5 and/or ITGA9, and/or the absence of cell surface markers CD45, CD321, CD56, JAM1 and/or ITGA7, or a selection of at least 1, at least 2, at least 3, at least 4, at least 5, at least 6, at least 7, at least 8, at least 9 or at least 10 of said cell surface markers. More preferably, FAPs are characterized by the presence of cell surface marker CD140a. FAPs may also be characterized by the presence of cell surface marker ITGA5, and/or the presence of cell surface marker CD140a, and the absence of cell surface marker ITGA7. For example, FAPS are pre-sorted according to Dohmen et al., Muscle-derived fibro-adipogenic progenitor cells for production of cultured bovine adipose tissue; 2022; npj Science of Food, 6, 6.

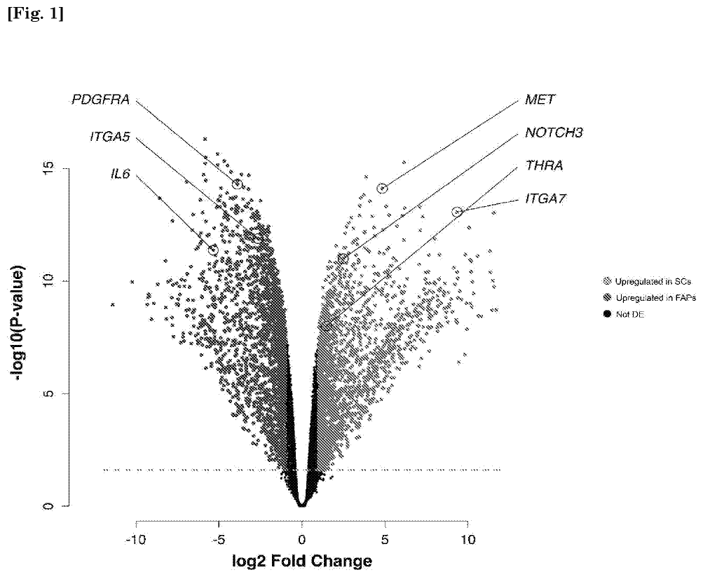

For example, myosatellite cells (SCs) are characterized by the presence of cell surface marker ITGA7. Alternatively, SCs are characterized by the absence of cell surface marker CD45, JAM1, and/or ITGA5, and/or the absence of cell surface marker CD140a and the presence of cell surface marker ITGA7. For example, FACS purification of FAPs and SCs is performed as shown in Example 1, FIG. 1A.

FAP cells and myosatellite cells may originate from the same (muscle) tissue sample, and are pre-sorted in the same antigen-based cell sorting procedure by employing cell surface markers that allow for these cell types to be distinguished.

### Culturing and Proliferating of a Muscle Progenitor Cell or an Adipogenic Progenitor Cell in the Selection Medium of the Present Invention

The present disclosure refers to a selection medium for culturing a muscle tissue sample, preferably a muscle tissue sample, comprising a muscle progenitor cell and an adipogenic progenitor cell, wherein the selection medium comprises at least one compound which specifically promotes the proliferation of the muscle progenitor cell or of the adipogenic progenitor cell. The method of the present disclosure comprises a step of culturing the tissue sample, preferably muscle tissue sample, in a selection medium of the present disclosure.

The culturing may be performed directly after provision of the tissue sample, preferably muscle tissue sample. Alternatively, the culturing may be performed after pre-processing and/or pre-sorting of the tissue sample as described above. The selection medium of the present disclosure specifically proliferates the muscle progenitor cell or the adipogenic progenitor cell, wherein the selection medium comprises at least one compound which specifically promotes the proliferation of the muscle progenitor cell or of the adipogenic progenitor cell.

For example, the selection medium of the present disclosure comprises at least one compound specifically promoting the proliferation of the muscle progenitor cell, preferably of the myosatellite cells. The at least one compound for specifically promoting the proliferation of the muscle progenitor cell is for example selected from the group consisting of fibroblast growth factor 2 (FGF-2), for example 10xFGF-2 (e.g. about 100 ng/ml), hepatocyte growth factor (HGF), for example 10xHGF (e.g. about 50 ng/ml), Triiodothyronine (T3) (e.g. about 30 nM),

Dexamethasone, Hydrocortisone (e.g. about 138 nM), Indomethacin (e.g. about 50 μM), SB203580 (4-{4-(4-Fluorophenyl)-2-[4-(methanesulfinyl)phenyl]-1 H-imidazol-5-yl} pyridine), Vitronectin, Laminin 521, and a combination thereof. For example, the compounds for specifically promoting the proliferation of the muscle progenitor cell comprise HGF, for example 10xHGF (e.g. about 50 ng/ml), and Triiodothyronine (T3) (e.g. about 30 nM) preferably also in the presence of FGF2.

As an example, the selection medium as disclosed herein preferably comprises an FGF-2 (e.g. about 100 ng/ml), a HGF (e.g. about 50 ng/ml) and a T3 (e.g. about 30 nM), optionally in the absence of a PDGF-bb. More preferably, the selection medium comprises an FGF-2 (e.g. about 100 ng/ml), a HGF (e.g. about 50 ng/ml), a T3 (e.g. about 30 nM) and a laminin such as laminin 521 (preferably as a support structure coating) optionally in the absence of a PDGF-bb.

Alternatively, the selection medium of the present disclosure comprises at least one compound specifically promoting the proliferation of the adipogenic progenitor cell, preferably a FAP. The at least one compound for specifically promoting the proliferation of the adipogenic progenitor cell is for example selected from the group consisting of PDGF-bb, for example 10xPDGF-bb (e.g. about 50 ng/ml), Interleukin 6 (IL-6) (e.g. about 20 ng/ml), Fibronectin, Collagen, and a combination thereof.

The selection medium is for example a suspension, a solution, or a colloid. The selection medium is designed to specifically promote the proliferation/growth of muscle progenitor cells, such as SCs, or of adipogenic progenitor cells, such as FAPs. This way the selection medium ensures the selective generation of either muscle progenitor cells, such as SCs, or of adipogenic progenitor cells, such as FAPs.

The at least one compound comprised in the selection medium of the present disclosure may be in solution or attached to a support structure. For example, the at least one compound is dissolved, diluted, diffused or dispersed in the liquid portion of the medium. The at least one compound may be soluble or insoluble in the liquid portion of the selection medium. The at least one compound may be in liquid, solid or gaseous form in the selection medium. The at least one compound is for example comprised in the selection medium by being attached and/or connected to a support structure, e.g. a culture vessel. For example, the at least one compound is attached to a support structure in form of a coating, i.e., coated on a culture vessel.

For example, Laminin 521 and/or Vitronectin is coated on a support structure, such as a culture vessel, for specifically promoting the proliferation of the muscle progenitor cells, such as SCs. For example, Fibronectin and/or Collagen is coated on a support structure, such as a culture vessel, for specifically promoting the proliferation of the adipogenic progenitor cells, such as FAPs.

The at least one compound specifically promoting the proliferation of the muscle progenitor cell or of the adipogenic progenitor cell is for example be provided in a concentration of from 0.1 nM-100 mM, 1 nM-10 mM, 10 nM-5 mM, 50 nM-1 mM, 100 nm-0.1 mM, 500 nM-1000 nM. Preferably, the concentration of the at least one compound of the present disclosure is 100 nM-1 mM. The at least one compound specifically promoting the proliferation of the muscle progenitor cell or of the adipogenic progenitor cell is for example provided of from −25% to +50% of the above given concentrations. Preferably, the proteins (e.g. growth factors and cytokines) as disclosed herein are recombinant human proteins.

The at least one compound specifically promoting the proliferation of muscle progenitor cells or adipogenic progenitor cells may be dissolved in the medium and/or coated on a support structure, e.g. a cultivation vessel. For example, in a method of the present disclosure for generating a homocellular cell culture of muscle progenitor the compounds HGF and T3 (and for instance FGF-2) are part of the liquid portion of the selection media and Laminin 521 is added to the selection medium in form of a coating on the culture vessel, e.g. flasks. For example the coating is applied as an aqueous solution, optionally wherein excess fluid is removed. For example, the coating is applied in the form of a solution, which is administered to the culture vessel and incubated at 37° C. for 90 minutes, during which time the coating adheres to the culture vessel, and the excess solution can be removed and replaced with culture medium. For example, the solution is phosphate-buffered saline (PBS) in water. For example, the solution is 20 mM acetic acid in water. For example, the solution is 20 mM acetic acid if collagen is applied as a coating.

The at least one compound specifically promoting the proliferation of muscle progenitor cells or adipogenic progenitor cells coated on a support structure, e.g. a cultivation vessel, is for example provided in an amount of from 0.01-100 μg/cm², 0.05-50 μg/cm², 0.1-25 μg/cm², 0.01-15 μg/cm², 0.2-10 g/cm², 0,25-8 μg/cm², 0.5-8 μg/cm², 1-1 μg/cm². For example, if the at least one compound is Laminin 521 coated on a support structure the amount can be from 0.25-4 μg/cm². For example, if the at least one compound is Fibronectin coated on a support structure the amount can be from 1-8 μg/cm². For example, if the at least one compound is Collagen coated on a support structure the amount can be from 0.5-6 μg/cm².

The selection medium of the present disclosure further comprises the essential nutrients, vitamins, growth factors, proteins, electrolytes, etc. which further support the survival and proliferation of the muscle progenitor cell or adipogenic progenitor cell. The selection medium is for example serum-free and does not comprise animal derived components. Preferably, the method of the present disclosure is entirely animal-component free. All components of the selection medium as well as the at least one compound specifically promoting proliferation of a muscle progenitor cell, such as SC, or of a adipogenic progenitor cell, such as FAP, may be not obtained by animals (animal-free).

The selection medium for specifically promoting adipogenic progenitor cells, such as FAPs, for example comprises DMEM/F12, Bovine Serum Albumin, a-linolenic acid, Vitamin C (2-phospho-L-ascorbic acid), ITSE, GlutaMax, Glucose, PSA, bFGF, Human IL-6, IGF-1, and PDGF-bb.

For example, the selection medium for specifically promoting adipogenic progenitor cells, such as FAPs, comprises DMEM/F12, Bovine Serum Albumin having a final concentration of 5.0 mg/ml, a-linolenic acid having a final concentration of 1.0 μg/ml, Vitamin C (2-phospho-L-ascorbic acid) having a final concentration of 50 μg/ml, 1 x ITSE, 1 x GlutaMax, Glucose having a final concentration of 17.7 mM, 1× PSA, bFGF having a final concentration of 10 ng/ml, Human IL-6 having a final concentration of 20 ng/ml, IGF-1 having a final concentration of 10 ng/ml and, PDGF-bb having a final concentration of 10 ng/ml.

Culturing the tissue sample, preferably muscle tissue sample, in a selection medium to specifically proliferate the muscle progenitor cell or the adipogenic progenitor cell is for example performed multi-dimensionally, such as 2-dimensionally, or 3-dimensionally. For two-dimensional culturing, a tissue sample or a heterocellular cell mixture comprising muscle progenitor cells and adipogenic progenitor cells may be propagated in tissue culture flasks in the selection medium promoting the proliferation of muscle progenitor cells or of adipogenic progenitor cells, respectively. For three-dimensional culturing, e.g. in suspension cultures, microcarrier-based cell cultures or aggregate-based cell cultures, a tissue sample or a heterocellular cell mixture comprising muscle progenitor cells and adipogenic progenitor cells may be propagated in any suitable vessel such as spinner flasks or bioreactors which may comprise microcarriers. Three-dimensional culturing is advantageous compared to two-dimensional culturing when aiming to achieve proliferation/expansion on a large scale. Preferably, in a method of the disclosed embodiments, culturing of a tissue sample or a heterocellular cell mixture comprising muscle progenitor cells and adipogenic progenitor cells is performed in a way that leads to three-dimensional proliferation/expansion.

For example, culturing a tissue sample or a heterocellular cell mixture comprising muscle progenitor cells and adipogenic progenitor cells to generate a homocellular cell culture of muscle progenitor cells, such as SCs, or of adipogenic progenitor cells, such as FAPs, is performed in the form of a microcarrier-based cell culture.

Exemplary culturing conditions for culturing a heterocellular tissue sample or a heterocellular cell mixture comprising muscle progenitor cells (SCs) and adipogenic progenitor cells (FAPs) according to the present disclosure are as follows. Cells, comprising muscle progenitor cells, such as SCs, and adipogenic progenitor cells, such as FAPs, are seeded at a density of 3000-5000 cells/cm² in the selection medium of the present disclosure, preferably being serum-free, in an appropriate cell culture vessel.

Optionally, the method comprises harvesting of the generated homocellular cell culture of muscle progenitor cells, such as SCs, or of adipogenic progenitor cells, such as FAPs. Harvesting is for example performed when the homocellular cell culture of muscle progenitor cells, such as SCs, or of adipogenic progenitor cells, such as FAPs is formed. For example, harvesting is performed when the generated homocellular cell culture of muscle progenitor cells, such as SCs, or of adipogenic progenitor cells, such as FAPs, has a percentage of muscle progenitor cells or of adipogenic progenitor cells from 90-100%, 92-99.9%, 93-99.7%, 94-99.5%, 95-99%, above 90%, above 93%, above 95%, above 97%, above 98%, above 99%, or above 99.5% referring to the total number of cells. The percentage is for example determined in the proliferation phase of the cell culture. Alternatively, harvesting is for example performed when the cell culture reached the culture vessels growth capacity and cell proliferation is repressed. Harvesting can be also performed after the cells are allowed to differentiate, by exposing them to an appropriate differentiation medium (outside the scope of this disclosed embodiments).

For example, for harvesting the cells can be rinsed once with phosphate buffer saline (PBS, 20012027 from ThermoFischer Scientific) followed by the addition of trypsin (25200072 from ThermoFischer Scientific). Once the cells are detached, trypsin can be neutralized by the addition of trypsin inhibitor from Glycine max (T6522 from Sigma Aldrich), the cells collected into PBS and centrifuged at 350 g. The supernatant can be aspirated and the cell pellet resuspended as needed. The cells can then be maintained in a 95% air/5% CO₂ humidified atmosphere at 37° C.

Muscle progenitor cells, such as SCs, or adipogenic progenitor cells, such as FAPs, of the generated homocellular cell cultures generated by the method of the present disclosure or by the use of the selection medium of the present disclosure may further be partially and/or terminally differentiated. An example of a partially differentiated muscle progenitor cell is a myoblast. An example of a terminally differentiated muscle progenitor cell is a myocyte, myotube and/or a myofiber. For example, a differentiated adipogenic progenitor cell is a adipocyte/fat cell. For example, the progenitor cells obtained by the present disclosure will be differentiated into muscle cells or fat cells, respectively.

Food products for animal or human consumption Muscle progenitor cells, such as SCs, or adipogenic progenitor cells, such as FAPs, of the homocellular cell cultures generated by the method of the present disclosure or by the use of the selection medium of the present disclosure may be used for producing a cell-culture based food product, such as a meat product and/or fat product. For example, a cell culture-based fat product does not contain muscle cells. The cell culture-based food product derived from the muscle progenitor cells, such as SCs, and/or from the adipogenic progenitor cells, such as FAPs, generated by the present disclosure may be for animal consumption, preferably for human consumption.

Non-limiting examples of a food product of the present disclosure are a hamburger, a sausage, a steak, minced meat, a meatball, corned beef, a charcuterie product, jerky or stewed meat. Food products also covers the combination of several types of meat products. The cultured fat cells as disclosed herein and optionally the muscle cells as disclosed herein are processed prior to or following incorporation into a food product. Non-limiting examples of processing are boiling, grilling, freezing, pressing, salting, curing, fermenting, smoking, drying, canning, cutting, grinding, mixing, seasoning, tubing in casing and marinating. The cultured fat cells and optional cultured muscle cells of the disclosed embodiments may be arranged in a specific manner in the food product, for example in order to create optical similarity with traditionally produced meat products and/or to improve texture.

A food product of the present disclosure may contain between 0.01% and 70% cultured fat cells, such as between 1% and 30% fat cells or between 5% and 20% fat cells. Additionally to fat cells and optionally muscle cells, meat products may comprise water, one or more salt, one or more fiber, one or more carbohydrate, one or more protein, one or more starch, one or more spice, one or more herb, one or more yeast extract, one or more casing ingredient, one or more vitamin, one or more oil, one or more hydrocolloid, one or more thickening agent, one or more preservative, one or more colorant, one or more antioxidant, one or more acidity regulator, one or more stabilizer, one or more emulsifier, one or more flavor enhancer and/or one or more sweetener. Preferably, all constituents of the food product are animal-free.

The food product of the present disclosure may be optically, structurally, in terms of flavor and/or in terms of composition identical or similar or corresponds to existing traditional meat products wherein animals are slaughtered in order to obtain said meat product. The food product of the present disclosure may have a composition that has beneficial characteristics in terms of human health and/or consumer preference when compared to existing traditional meat products wherein animals are slaughtered in order to obtain said meat product.

Optionally, the food product of the present disclosure comprises more unsaturated fatty acids compared to bovine(-derived) subcutaneous fat tissue. Unsaturated fatty acids are generally more beneficial to animal health, more preferably human health, compared to saturated fatty acids. Optionally, the food product of the disclosed embodiments comprises no inflammatory cells. Existing traditional meat products are made from fat tissue and muscle tissue from the animal body, and therefore comprise inflammatory cells. Optionally, the food product of the disclosed embodiments comprises fewer, preferably no, antibiotics and/or antibiotics residues. Antibiotics in food products are a burden to animal health, including human health, when consumed, as they may kill part of the animal gut microbiome. Also, the presence of antibiotics in food may allow for the promotion of antibiotic resistance. Furthermore, antibiotics may lead to tissue damage, for example in the animal gut.

The food product of the present disclosure may comprise no blood residues such as red blood cells. Blood components may lead to lipid oxidation and may decrease the shelf life of the food products. Existing traditional meat products generally contain blood components such as red blood cells.

Optionally, the food product of the present disclosure comprises lower levels of microbial contamination compared to traditional meat products. The food product of the present disclosure is preferably produced in controlled environments that aim to prevent contamination with microbials. Also, since significantly less animal tissue is used in the production of the food product of the disclosed embodiments compared to traditional meat products, potential microbials present in or on animal tissue are incorporated in a meat product in much lower levels compared to traditional meat products.

Optionally, the food product of the present disclosure does not comprise cartilage. Cartilage may have a negative effect on the consumption experience of the consumer, as it is much tougher than (artificial) muscle tissue or (artificial) fat tissue. Absence of cartilage in a food product is generally associated with a higher quality.

Optionally, the food product of the present disclosure comprises lower levels of fibrous tissue compared to traditional meat and/or fat products. Fibrous tissue, otherwise referred to as connective tissue, comprises proteins such as collagen and elastin that render meat tough and therefore less beneficial for consumption. Fibrous tissue may be associated with lower quality food products. Lower levels of fibrous tissue are also beneficial in the preparation of food products, as it generally takes less time to cook food products with low levels if fibrous tissue.

### Numbered Embodiments

Embodiment 1. Method for specifically generating a homocellular cell culture of muscle progenitor cells or of adipogenic progenitor cells from a heterocellular muscle tissue sample comprising the steps of:
a) Providing a muscle tissue sample comprising at least one muscle progenitor cell and at least one adipogenic progenitor cell;
b) Culturing the muscle tissue sample in a selection medium to specifically proliferate the at least one muscle progenitor cell or the at least one adipogenic progenitor cell, wherein the selection medium comprises at least one compound which specifically promotes the proliferation of the at least one muscle progenitor cell or of the at least one adipogenic progenitor cell; and
c) Optionally, harvesting the generated homocellular cell culture.

Embodiment 2. Method according to embodiment 1, further comprising the steps of:
a-1) enzymatically pre-processing the heterocellular muscle tissue sample for obtaining a heterocellular cell mixture, and/or
a-2) pre-sorting the muscle progenitor cell and/or the adipogenic progenitor cell of the heterocellular muscle tissue sample, wherein both steps are performed prior the culturing of step b).

Embodiment 3. Method according to embodiment 2, wherein the pre-sorting of step a-2) is performed by antigen-based cell sorting, preferably by fluorescence activated cell sorting (FACS).

Embodiment 4. Method according to any one of the preceding embodiments, wherein the generated homocellular cell culture of muscle progenitor cells or of adipogenic progenitor cells has a percentage of muscle progenitor cells or of adipogenic progenitor cells from 90% to 100%, preferably from 95% to 100%, more preferably above 97% referring to the total number of cells.

Embodiment 5. Method according to embodiment 4, wherein the percentage of muscle progenitor cells or of adipogenic progenitor cells is determined in the proliferation phase.

Embodiment 6. Method according to any one of the preceding embodiments, wherein the at least one compound specifically promoting the proliferation of the muscle progenitor cell is selected from the group consisting of fibroblast growth factor 2 (FGF-2), hepatocyte growth factor (HGF), Triiodothyronine (T3), Dexamethasone, Hydrocortisone, Indomethacin, SB203580 (4-{4-(4-Fluorophenyl)-2-[4-(methanesulfinyl)phenyl]-1 H-imidazol-5-yl} pyridine), Vitronectin, Laminin 521, and a combination thereof.

Embodiment 7. Method according to any one of embodiments 1 to 5, wherein the at least one compound specifically promoting the proliferation of the adipogenic progenitor cell is selected from the group consisting of PDGF-bb, Interleukin 6 (IL-6), Fibronectin, Collagen, and a combination thereof.

Embodiment 8. Method according to any one of the preceding embodiments, wherein the at least one compound is comprised in the selection medium in solution or attached to a support structure.

Embodiment 9. Method according to any one of the preceding embodiments, wherein at least one compound is coated on a culture vessel.

Embodiment 10. Method according to any one of the preceding embodiments, wherein the culturing of step b) further comprises culturing the muscle tissue sample in a culture vessel which is coated with at least one of the compounds specifically promoting the proliferation of the muscle progenitor cell or of the adipogenic progenitor cell.

Embodiment 11. Method according to any one of embodiments 1 to 6, and 8 to 10, wherein the culturing of step b) is performed using a selection medium comprising HGF (preferably about 50 ng/ml), T3 (preferably about 30 nM T3) and Laminin 521 for specifically proliferating muscle progenitor cells, wherein Laminin 521 is coated on a culture vessel.

Embodiment 12. Method according to any one of the preceding embodiments, wherein the muscle progenitor cell is a myosatellite cell (SC) and/or the adipogenic progenitor cell is a fibro-adipogenic progenitor cell (FAP).

Embodiment 13. Method according to any one of the preceding embodiments, wherein the heterocellular muscle tissue sample is from mammalian, preferably from bovine, ovine, murine, or porcine origin, more preferably from bovine origin.

Embodiment 14. Method according to any one of the preceding embodiments for producing a cell culture-based meat product, preferably for animal or human consumption.

Embodiment 15. Selection medium for culturing a muscle tissue sample comprising a muscle progenitor cell and an adipogenic progenitor cell, wherein the selection medium comprises at least one compound which specifically promotes the proliferation of the muscle progenitor cell or of the adipogenic progenitor cell.

Embodiment 16. Selection medium according to embodiment 15, wherein the at least one compound specifically promoting the proliferation of the muscle progenitor cell is selected from the group consisting of fibroblast growth factor 2 (FGF-2), hepatocyte growth factor (HGF), Triiodothyronine (T3), Dexamethasone, Hydrocortisone, Indomethacin, SB203580, Vitronectin, Laminin 521, and a combination thereof.

Embodiment 17. Selection medium according to embodiment 15 or embodiment 16, wherein the selection medium comprises HGF (preferably about 50 ng/ml HGF) and T3 (preferably about 30nM T3) for specifically promoting the proliferation of the muscle progenitor cell.

Embodiment 18. Selection medium according to embodiment 15, wherein the at least one compound specifically promoting the proliferation of the adipogenic progenitor cells is selected from the group consisting of PDGF-bb, Interleukin 6 (IL-6), Fibronectin, Collagen, and a combination thereof.

Embodiment 19. Selection medium according to any one of the preceding embodiments, wherein the at least one compound comprised in the selection medium is in solution or attached to a support structure, e.g., coated on a culture vessel.

Embodiment 20. Selection medium according to any one of the preceding embodiments, wherein the selection medium is serum-free.

Embodiment 21. Use of the selection medium according to any one of embodiments 15 to 20 for specifically generating a homocellular cell culture of muscle progenitor cells or of adipogenic progenitor cells from a heterocellular muscle tissue sample, preferably from a heterocellular bovine muscle tissue sample.

### EXAMPLES

### Example 1: Identification of Compounds to Favor SC or FAP Growth

### Materials & Methods

### Bovine Muscle Biopsy

Animal health check was performed by a veterinarian prior to biopsy. Animals were sedated (Xyla-Ject 2%, 0.15 ml/100 kg) via the tail vein. Local anesthetic (Procamidor, 20 mg/mL) was applied to the biopsy site via subcutaneous injection. Muscle was exposed by creating an incision in the skin using a scalpel, and approximately one gram of skeletal muscle tissue collected on ice. Wound was closed using skin sutures (PGA 6/0) and covered with aluminum spray. Analgesic (Novem 20, 0.025 mL/kg) was applied subcutaneously. Post-biopsy health checks were performed daily by the farmer for 10 days post-procedure.

### Isolation of Bovine Muscle-Derived Cells

Excess visible fat and fibrous tissue was removed prior to dissociation, and muscle fibers were dissociated using collagenase AFC A (Worthington, CLSAFA, 2000U/ml) for 45 minutes at 37° C. Muscle isolates were incubated in 1× ACK erythrocyte lysis buffer for 1 minute at room temperature. Cells were resuspended in a serum-free proliferation medium (SFM1 medium) and immediately seeded into fibronectin (4 μg cm-2 bovine fibronectin; F1141, Merck/Sigma) coated tissue culture plates, and pre-cultured at 37° C. for 72 hours. The serum-free proliferation medium that was used is as disclosed in WO 2021/158103, the contents of which are incorporated herein by reference, and is referred to therein as “SFM1” and contains: albumin (5 mg/ml), somatotropin (2 ng/ml), L-Ascorbic acid 2-phosphate (50 μg/ml), hydrocortisone (36 ng/ml), a-linolenic acid (1 μg/ml), insulin (10 μg/ml), transferrin (5.5 μg/ml), sodium selenite (0.0067 μg/ml), ethanolamine (2 μg/ml), L-alanyl-L-glutamine or glutamine (2 mM), IL-6 (5 ng/ml), FGF2 also referred to as bFGF (10 ng/ml), IGF1 (100 ng/ml), VEGF (10 ng/ml), HGF (5 ng/ml), PDGF-bb (10 ng/ml) and DMEM/F12 basal medium. In the experiments described herein, the SFM1 medium is also referred to as control (Ctrl) or base medium.

### FACS Purification of FAP Cells and SC Cells

After 72 hours of pre-culture, FAPS were sorted using a MACSQuant Tyto sorter (Miltenyi) based on absence of expression of JAM1, CD45, and integrin alpha 7 (ITGA7), and the positive expression of integrin alpha 5 (ITGA5). SCs were sorted based on the absence of expression of JAM1, CD45, ITGA5, and the positive expression of ITGA7.

### Proliferation of FAP and SC Cells

After FACS sorting, FAP cells and SC cells were cultured on collagen Type I coated tissue culture flasks at 37° C. and 5% CO2 in a serum-free proliferation medium (SFM1). Purities of SCs and FAPs were assessed at the end of each passage by measuring the expression of ITGA7 and ITGA5 via flow cytometry as described below.

### RNA Sequencing

For RNA-sequencing (RNAseq), FAPs and SCs from two donor animals were sorted into TRK Lysis buffer. RNA was isolated using the Omega MicroElute Total RNA Kit (Omega Bio-tek), and sequenced on a NextSeq 500 after library preparation using the TruSeq stranded mRNA kit (Illumina). Resulting reads were aligned to the bosTau9 (ARS UCD1.2.98) reference genome and counted using the Rsubread package. Gene counts were normalized by normalization factors computed through the trimmed-mean of M-values. Samples were clustered using principal component analysis, and differentially expressed genes between FAPs and SCs were computed using empirical Bayes moderation of the standard error with the R-package limma.

### Flow Cytometry

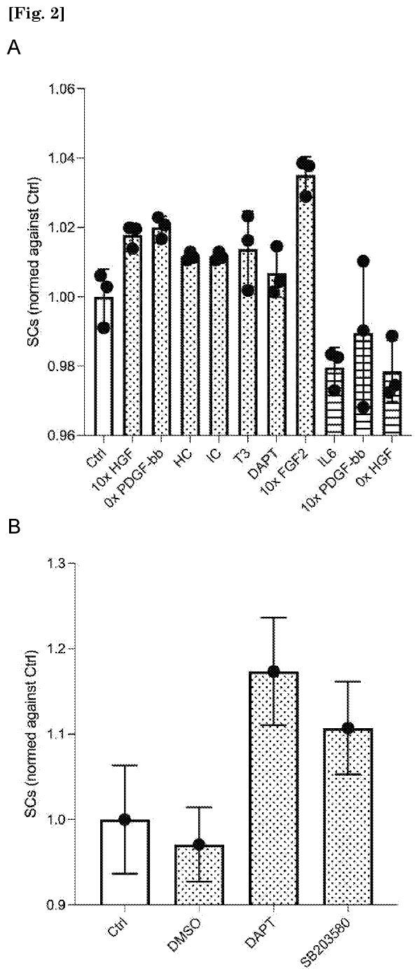

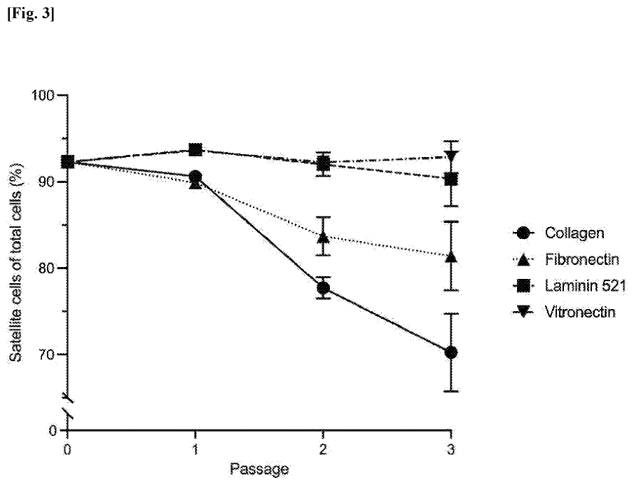

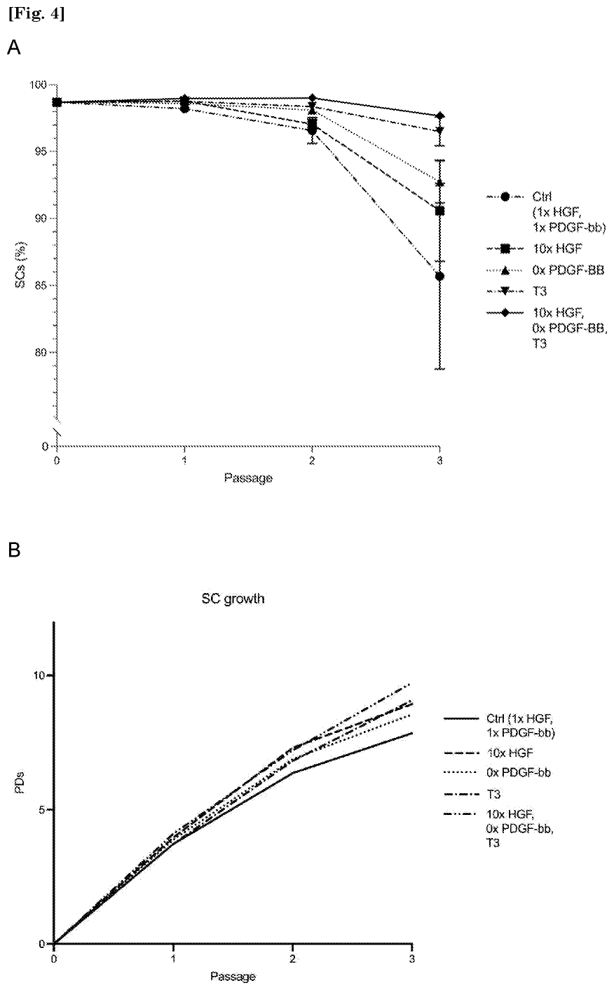

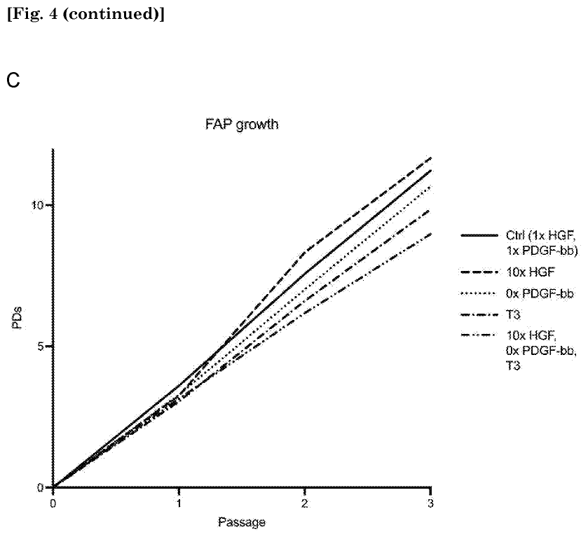

Sorted SCs and FAPs were stained with ITGA7-PE and ITGA7-APC. Subsequently, cells were washed and analyzed on a MACSQuant 10 flow analyzer (Miltenyi). Unstained cells were used as a negative control, and to define gating parameters. For the compound screenings (FIG. 2A), percentage of SCs relative to FAPs was determined after one culture passage.

### Results

Using bulk RNAseq to calculate differentially expressed genes between FAPs and SCs, we identified surface receptors that were expressed at significantly higher levels in either FAPs or SCs (FIG. 1). These informed the selection of compounds to favor the proliferation of either of the two cell types. Increasing or decreasing the concentration of growth factors targeting these receptors, or adding compounds that target downstream pathways, altered the growth of SCs relative to FAPs (FIG. 2A, FIG. 2B). As only SCs and FAPs were present in the cell cultures of FIG. 2A and 2B, measuring the relative amount (ratio) of SCs likewise provides information about the relative amount (ratio) of FAPs in the respective media. The ratio of SCs in the respective cell media was normalized against a control ratio (Ctrl; set at 1) which means that a promotion of SCs results in an increase of SCs compared to the control culture (>1), whereas a relative decrease of SCs in the medium compared to the control culture (<1) denotes at the same time for a promotion of FAP proliferation in the respective media.

It was established that certain individual compounds specifically promoted either SCs proliferation or FAPs proliferation. FIGS. 2A and 2B show that the addition of HGF, HC, IC, T3, DAPT, FGF2 or SB203580 specifically promoted the proliferation of SCs, whereas IL-6 and PDGF-bb specifically promoted the proliferation of FAPs (decrease of SCs). Hence, FIGS. 2A and 2B show clearly that the individual presence of certain compounds facilitates the promotion of either SC or FAP proliferation according to the present disclosure.

### Example 2: Growth Conditions Favor Specific Growth of SC or FAP in Long-Term Proliferation

### Materials & Methods

### Proliferation of FAP and SC Cells

Cells were isolated, purified and cultured as described in Example 1. For long-term proliferation assays, mixtures of SCs and FAPs were cultured on different coatings (FIG. 3) or on Laminin 521 with different media (FIGS. 4A-4C). After every passage, the ratio of SCs to FAPs was measured as the ratio of ITGA7+/ITGA5−cells (SCs) to ITGA7−/ITGA5+cells (FAPs) via flow cytometry, as described in Example 1.

### Results

Using long-term proliferation assays, combined with flow cytometry for ITGA7/ITGA5, we tested different compounds including extracellular matrix proteins tissue culture vessel coatings, and measured percentage of SCs over time in a cell culture that contains SCs and FAPs. On collagen or fibronectin, the proportion of FAPs increased quickly over 3 passages, whilst on Laminin 521 or vitronectin, the percentage of SCs remained high (see FIG. 3). Hence, collagen and fibronectin each represent compounds for specifically promoting FAP proliferation, while Laminin 521 and vitronectin each represent specific compounds for specifically promoting SC proliferation.

Using Laminin 521 as a coating, we further used the previously identified compounds to adapt the base media (Ctrl) to favor the growth of SCs over FAPs (FIGS. 4A-4C). We found that while each single media adaptation (10× HGF, 0× PDGF-bb, T3) already promoted the relative growth/proliferation of SCs compared to FAPs, the combination of all three adaptations, i.e., the presence of HGF and T3 and the absence of PDGF-bb, maximally increased SC growth/proliferation and slowed down/delayed FAP growth/proliferation.

1. Method for specifically generating a homocellular cell culture of muscle progenitor cells or of adipogenic progenitor cells from a heterocellular muscle tissue sample comprising the steps of:
a. Providing a muscle tissue sample comprising at least one muscle progenitor cell and at least one adipogenic progenitor cell;
b. Culturing the muscle tissue sample in a selection medium to specifically proliferate the at least one muscle progenitor cell or the at least one adipogenic progenitor cell,
wherein the selection medium comprises at least one compound in which specifically promotes the proliferation of the at least one muscle progenitor cell, wherein the at least one compound is in solution and is selected from the group consisting of fibroblast growth factor 2 (FGF-2), hepatocyte growth factor (HGF), Triiodothyronine (T3), Dexamethasone, Hydrocortisone, Indomethacin, SB203580 (4-{4-(4-Fluorophenyl)-2-[4-(methanesulfinyl)phenyl]-1H-imidazol-5-yl} pyridine), and/or the at least one compound is attached to a support structure in the form of a coating, when it is Vitronectin, Laminin 521; and a combination thereof, or
wherein the selection medium comprises at least one compound which specifically promotes the proliferation of the at least one adipogenic progenitor cell wherein the at least one compound is in solution and is selected from the group consisting of PDGF-bb, Interleukin 6 (IL-6), and/or the at least one compound is attached to a support structure in the form of a coating, when it is Fibronectin, Collagen;; and a combination thereof; and
c. Optionally, harvesting the generated homocellular cell culture.

2. Method according to claim 1, further comprising the steps of:
a-1) enzymatically pre-processing the heterocellular muscle tissue sample for obtaining a heterocellular cell mixture, and/or
a-2) pre-sorting the muscle progenitor cell and/or the adipogenic progenitor cell of the heterocellular muscle tissue sample, wherein both steps are performed prior the culturing of step b).

3. Method according to claim 2, wherein the pre-sorting of step a-2) is performed by antigen-based cell sorting, preferably by fluorescence activated cell sorting (FACS).

4. Method according to claim 1, wherein the generated homocellular cell culture of muscle progenitor cells or of adipogenic progenitor cells has a percentage of muscle progenitor cells or of adipogenic progenitor cells from 90% to 100%, preferably from 95% to 100%, more preferably above 97% referring to the total number of cells.

5. Method according to claim 4, wherein the percentage of muscle progenitor cells or of adipogenic progenitor cells is determined in the proliferation phase.

6-7. (canceled)

8. Method according to claim 1, wherein the culturing of step b) further comprises culturing the muscle tissue sample in a culture vessel which is coated with at least one of the compounds specifically promoting the proliferation of the muscle progenitor cell or of the adipogenic progenitor cell.

9. Method according to claim 1, wherein the culturing of step b) is performed using a selection medium comprising HGF, T3 and Laminin 521 for specifically proliferating muscle progenitor cells, wherein Laminin 521 is coated on a culture vessel.

10. Method according to claim 1, wherein the muscle progenitor cell is a myosatellite cell (SC) and/or the adipogenic progenitor cell is a fibro-adipogenic progenitor cell (FAP).

11. Method according to claim 1, wherein the heterocellular muscle tissue sample is from mammalian, preferably from bovine, ovine, murine, or porcine origin, more preferably from bovine origin.

12. Method according to claim lany one of the preceding claims for producing a cell culture-based meat product, preferably for animal or human consumption.

13. Selection medium for culturing a muscle tissue sample comprising a muscle progenitor cell and an adipogenic progenitor cell,
wherein the selection medium comprises at least one compound which specifically promotes the proliferation of the muscle progenitor cell, wherein the at least one compound is in solution and selected from the group consisting of fibroblast growth factor 2 (FGF-2), hepatocyte growth factor (HGF), Triiodothyronine (T3), Dexamethasone, Hydrocortisone, Indomethacin, SB203580, or attached to a support structure in the form of a coating, when it is Vitronectin or Laminin 521; and a combination thereof; or
wherein the selection medium comprises at least one compound which specifically promotes the proliferation of the adipogenic progenitor cell, wherein the at least one compound is in solution and selected from the group consisting of PDGF-bb, Interleukin 6 (IL-6), or attached to a support structure in the form of a coating, when it is Fibronectin or Collagen; and a combination thereof.

14. Selection medium according to claim 13, wherein the selection medium comprises HGF and T3 for specifically promoting the proliferation of the muscle progenitor cell.

15. (canceled)

16. Selection medium according to claim 13, wherein the selection medium is serum-free.

17. Use of the selection medium according to claim 13 for specifically generating a homocellular cell culture of muscle progenitor cells or of adipogenic progenitor cells from a heterocellular muscle tissue sample, preferably from a heterocellular bovine muscle tissue sample.
